SEQUENCE LISTING (3 sequences)

SEQ ID NO: 1 - DNA, 708 bp, homosapiens
  CDS (1)..(708)
atggattttcaggtgcagattatcagcttcctgctaatcagtgcttcagtcataatgtccagaggacaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacttcttactctctcaccatcagcagagtggaggctgaagatgctgccacttattactgccagcagtggactagtaacccacccacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 2 - protein, 235 aa, homosapiens
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1413 bp, homo sapiens
  CDS (1)..(1413)
atgggctggagtctgattctgctcttcctggtggctgtggccacaagagtgctgtcccaggtccagctgcagcagctgggggccgagctggttaaacctggcgcctccgtgaagatgtcctgcaaagcatccggctacaccttcaccagttacaacatgcattgggtgaagcaaactccgggcagaggcctggaatggataggggcgatctaccccgggaacggtgacacgagttacaaccagaagttcaagggcaaggcgaccctcaccgccgacaagtcgtcctcgaccgcttacatgcagctgtcctccctgacgtcggaggactcggccgtgtactactgcgcccgctcgacctactacggcggcgactggtacttcaacgtgtggggggccggcaccaccgtgaccgtgagtgccgcctccaccaaggggccgtccgtgttcccactcgcaccatcatccaagtccaccagtgggggcactgccgcattagggtgcctggtcaaggattactttcccgaacctgtgaccgtctcttggaattccggggccctcacgtccggggtccataccttcccagctgtgttgcagtcgtcggggctgtactccctgtcctcggttgttaccgtcccctcgtcaagtctgggcacccagacctacatctgcaacgtcaatcacaagccctcgaacaccaaggtcgacaagaaggccgaacccaagtcgtgcgacaagacgcacacatgtcctccctgccccgctcccgaactgctgggcggacctagtgtgttcctgttccctccaaagcccaaggacaccctcatgatctcacgcaccccagaggtgacctgcgtcgtcgtggatgtgtcgcacgaggatcccgaggttaagttcaactggtacgtggacggcgtggaggttcacaacgctaaaaccaagccacgggaggagcagtataactccacgtaccgcgtggtgtcagtgctcacagtgcttcaccaagactggctcaacgggaaggagtacaagtgcaaggtgtccaacaaggcccttcccgcccccatcgagaagactatctccaaggctaagggacagccgcgagagcctcaggtgtacaccctgcctccctcgcgcgacgagttaacaaagaaccaggtatcgctcacttgcctggtcaaaggtttctacccttccgacatcgccgtcgagtgggagagtaacgggcagcccgagaacaactataagactacaccccccgtgctagattccgatgggtccttcttcctgtactcgaagctcacagtggataagagtcgctggcagcaggggaacgtcttcagttgctccgtgatgcacgaggccctgcacaaccactacacgcagaagtccctgagcctgtcgcccggcaagtga

(19) United States
(12) Patent Application Publication
(10) Pub. No.: US 2009/0285795 A1
(43) Pub. Date: Nov. 19, 2009

(54) Method for the Production of a Monoclonal Antibody to CD20 for the Treatment of B-Cell Lymphoma

(76) Inventor: Villoo Morawala Patell, Bangalore (IN)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/914,750

(22) PCT Filed: May 24, 2006
(86) PCT No.: PCT/IB2006/001358

§ 371 Date: Nov. 18, 2007

(30) Foreign Application Priority Data
May 24, 2005 (IN) 624/CHE/2005

(51) Int. Cl. A61K 39/395; C12P 21/04

(52) U.S. Cl. 424/130.1; 435/69.6

(57) ABSTRACT

The present invention relates to the recombinant method used for the production of soluble form of an antibody that binds to CD20 for treatment of patients with relapsed or refractory, low-grade or follicular, CD20-positive, B-cell non-Hodgkin's lymphoma (NHL). The treatment will comprise the use of immunologically active anti-CD20 antibodies; or radiolabeled anti-CD20 antibodies and or cooperative strategies where both labeled and non-labeled antibodies will be used for treatment of NHL. The procedure describes the de novo synthesis of the nucleic acid sequence encoding anti-CD20, transformation of the constructed nucleic acid sequences into competent bacteria and the sub-cloning of the same into mammalian expression vectors for expression of the desired protein. DNA constructs comprising the control elements associated with the gene of interest has been disclosed. The nucleic acid sequence of interest has been codon optimized to permit expression in the suitable mammalian host cells.

### FIELD OF THE INVENTION

The present invention relates to the recombinant method used for the production of soluble form of an antibody that binds to CD20 for treatment of patients with relapsed or refractory, low-grade or follicular, CD20-positive, B-cell non-Hodgkin's lymphoma (NHL). The treatment will comprise the use of immunologically active anti-CD20 antibodies; or radiolabeled anti-CD20 antibodies and or cooperative strategies where both labeled and non-labeled antibodies will be used for treatment of NHL. The procedure describes the de novo synthesis of the nucleic acid sequence encoding anti-CD20, transformation of the constructed nucleic acid sequences into competent bacteria and the sub-cloning of the same into mammalian expression vectors for expression of the desired protein. DNA constructs comprising the control elements associated with the gene of interest has been disclosed. The nucleic acid sequence of interest has been codon optimized to permit expression in the suitable mammalian host cells.

### BACKGROUND OF THE INVENTION

Antibodies have been considered as a powerful tool to recognize and target almost any molecule with a high degree of specificity and affinity. Monoclonal antibodies (mAbs) have been used as in vitro diagnostics hence enabling worldwide standardisation of reagents for RIA, ELISA immunocytopathology and flow cytometry. Monoclonal antibodies have also been extensively used for in vivo localisation of tumour antigens and in the immunotherapy of cancer.

This problem has been addressed by the development of antibodies of two basic types. The first type, referred to as chimeric antibodies, wherein the murine constant domains only are replaced by equivalent domains of human origin (Morrison et al, P.N.A.S., 1984, 81, 6851-6855; Boulianne et al, Nature, 1985, 314, 268-270; and Neuberger et al, Nature, 1985, 314, 268-270). The second type is where the murine constant domains and the murine framework regions are all replaced by equivalent domains and regions of human origin. This second type of antibody is referred to as a humanized or CDR-grafted antibody (Jones et al, Nature, 1986, 321, 522-525; and Riechmann et al, Nature, 1988, 332, 323-327). For example, for therapeutic purposes, human IgG1 and rat IgG2b are currently favored isotypes. Further, of the human IgG isotypes,IgG1 and IgG3 appear to be most effective for complement and cell mediated lysis, and therefore for killing tumour cells. A human antibody would of course avoid the need for “humanization”, however cell lines, which secrete human antibodies, are very unstable and have generally proven unsuitable for commercial scale production.

To generate sufficient quantities of antibody for full clinical use it is desirable to employ an efficient recombinant expression system. Since myeloma cells represent a natural host specialized for antibody production and secretion, cell lines derived from these have been used for the expression of recombinant antibodies. Often, complex vector design, based around immunoglobulin gene regulatory elements, is required, and final expression levels have been reported which are highly variable (Winter et al, Nature, 1988, 332, 323-327; Weidle et al, Gene, 1987, 60, 205-216; Nakatani et al, Bio/Technology, 1989, 7, 805-810; and Gillies et al, Bio/Technology, 1989, 7, 799-804). An alternative mammalian expression system is that offered by the use of Chinese hamster ovary (CHO) cells. The use of these cells has enabled the production of large quantities of several therapeutic proteins for research and clinical use (Kaufman et al, Mol. Cell. Biol, 1985, 5, 1750-1759; and Zettlmeissl et al, Bio/Technology, 1987, 5, 720-725). There are, however, very few instances of the use of these cells for the expression of antibodies and the levels of expression of murine antibodies reported to date are low of the order of 0.01-0.1. mu.g/ml (Weidle et al, Gene, 1987, 51, 21-29; and Feys et al, Int. J. Cancer, 1988, 2, 26-27).

The anti-CD20 antibody binds specifically to the antigen CD20 (human B lymphocyte restricted differentiation antigen, Bp35), a hydrophobic transmembrane protein with a molecular weight of approximately 35 kD located on pre B and mature B lymphocytes. The antigen is also expressed on >90% of B cell non Hodgkin's lymphomas (NHL), but is not found on hematopoietic stem cells, pro B cells, normal plasma cells or other normal tissues. CD20 regulates an early step(s) in the activation process for cell cycle initiation and differentiation, and possibly functions as a calcium ion channel. CD20 is not shed from the cell surface and does not internalize upon antibody binding. Free CD20 antigen is not found in the circulation. The anti-CD20 antibody works by recruiting the body's natural defenses to attack and kill the B cell to which it binds via the CD20 antigen. The host cell killing takes place by two mechanisms: 1) the complement-dependent cytotoxicity (CDC) pathway and 2) the antibody dependent cell mediated cytotoxicity (ADCC) pathway.

The anti-CD20 antibody is a genetically engineered chimeric murine/human monoclonal antibody. The antibody is an IgG₁ kappa immunoglobulin containing murine light- and heavy-chain variable region sequences and human constant region sequences. The anti-CD20 antibody is composed of two heavy chains of 451 amino acids and two light chains of 213 amino acids (based on cDNA analysis) and has an approximate molecular weight of 145 kD. The anti-CD20 antibody has a binding affinity for the CD20 antigen of approximately 8.0 nM. The chimeric anti-CD20 antibody is produced by mammalian cell (Chinese Hamster Ovary) suspension culture in a nutrient medium containing the antibiotic gentamicin. The anti-CD20 antibody is purified by affinity and ion exchange chromatography.

The present invention relates to the construction, cloning, expression, purification and production of antibodies that can bind to CD20. The antibody will be a targeted therapy indicated for the treatment of patients with relapsed or refractory, low-grade or follicular, CD20-positive, B-cell non-Hodgkin's lymphoma (NHL).

### SUMMARY OF THE INVENTION

The present invention is directed to the transformation of nucleic acid sequence encoding the polypeptide anti-CD20.

According to an aspect of the invention there is provided the nucleic acid sequences encoding the heavy and the light chains of the anti-CD 20 molecule. According to an further aspect of the invention there is provided the corresponding amino-acid sequence encoded by the nucleic acid sequences.

A particular aspect of the invention relates to the de novo synthesis of the variable regions of the heavy and the light chain of the anti-CD 20 molecule. Further disclosed is the construction of the vector constructs with the nucleic acid sequence of interest, transformation of the vector constructs into competent bacteria and subcloning of the anti-CD 20 chains into mammalian expression vectors.

### DETAILED DESCRIPTION OF THE INVENTION

The anti-CD20 antibody is a mouse-human chimeric antibody. It is comprised of two chains—1) the light chain which is made up of the variable domain derived form the light chain of the mouse monoclonal antibody 2B8 and the human kappa constant domain and 2) the heavy chain which is made of the variable domain of the heavy chain of the mouse monoclonal antibody 2B8 and the human IgG1 constant domain. The antibody sequence is delineated in the patent WO 94/11026.

A de novo approach has been followed in terms of synthesis of the coding regions of cDNA-construct because the hybridoma 2B8 that secretes the anti-CD20 mouse monoclonal antibody is not available in the public domain. De novo gene synthesis would also enable codon optimization with respect to the particular mammalian cell line to be used for protein expression.

The nucleotide sequence encoding the light chain of the anti-CD20 antibody has been represented in SEQ ID 1.

The Codon-optimized version of the nucleotide sequence encoding the light chain of the anti-CD20 antibody has been represented in SEQ ID 2.

The codons in the coding DNA sequence of the light chain of the anti-CD20 antibody that have been altered as part of the codon-optimization process to ensure optimal recombinant protein expression in mammalian cell lines such as CHO K1 and HEK 293. The Nucleotide sequence encoding the heavy chain of the anti-CD20 antibody has been represented in SEQ ID 3.

The Codon-optimized version of the nucleotide sequence encoding the heavy chain of the anti-CD20 antibody has been depicted in SEQ ID 4.

The codons in the coding DNA sequence of the heavy chain of the anti-CD20 antibody that have been altered as part of the codon-optimization process to ensure optimal recombinant protein expression in mammalian cell lines such as CHO K1 and HEK 293.

### Choice of the Expression Vector:

The design of the mammalian expression vector for the expression of the anti-CD20 antibody can be based on one of the commercially available vectors (eg: pcDNA or pIRES from Invitrogen or BD Biosciences respectively), modified to include the following features:

(a) A multiple cloning site for insertion of cDNA encoding both the light and the heavy antibody chains of the anti-CD20 antibody in separate expression cassettes in the same vector.

(b) The design of the expression vector can also accommodate an independent (bi-cistronic) IRES-mediated co-expression of the green fluorescent protein which would allow rapid screening of highly expressing transfectants using fluorescence assisted cell sorting.

(c) Cloning of the chimeric light and heavy antibody chains in two separate mammalian expression systems having different selectable markers.

### Sub-Cloning of the RTX Antibody Chains in Mammalian Expression Vectors

The de novo synthesized anti-CD20 antibody-heavy chain (RTX-HC) and anti-CD20 antibody-light chain (RTX-LC) were obtained as cDNA fragments cloned into the pBSKII vector and construct referred as pBSKII-RTX-LC and pBSKII-RTX-HC respectively. The DNA was transformed into DH10b E. coli cells and plated onto LB agar plates containing ampicillin. A colony from the plate was inoculated in liquid medium and a DNA mini prep was carried out. The sequence of the two antibody chains was confirmed by sequencing.

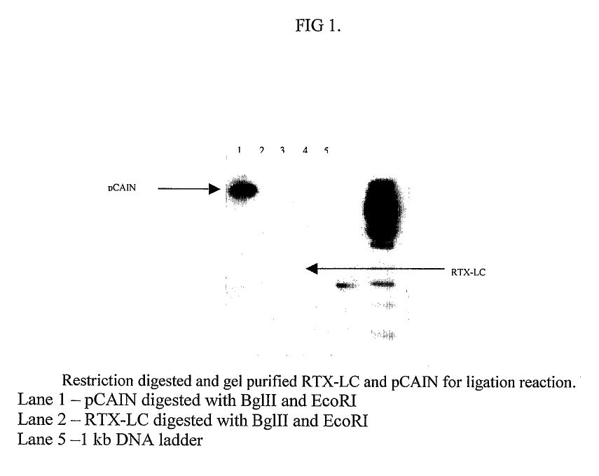

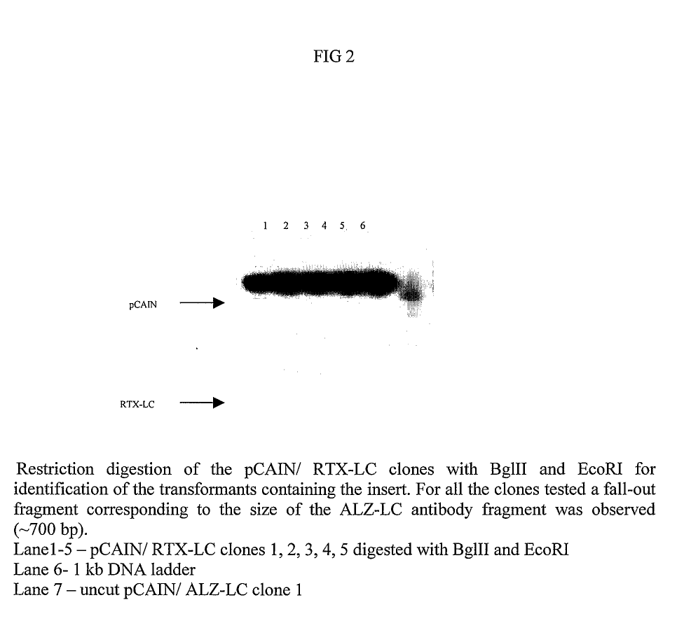

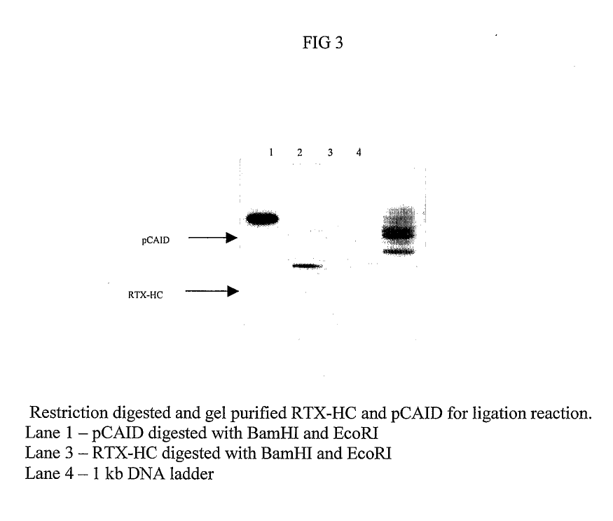

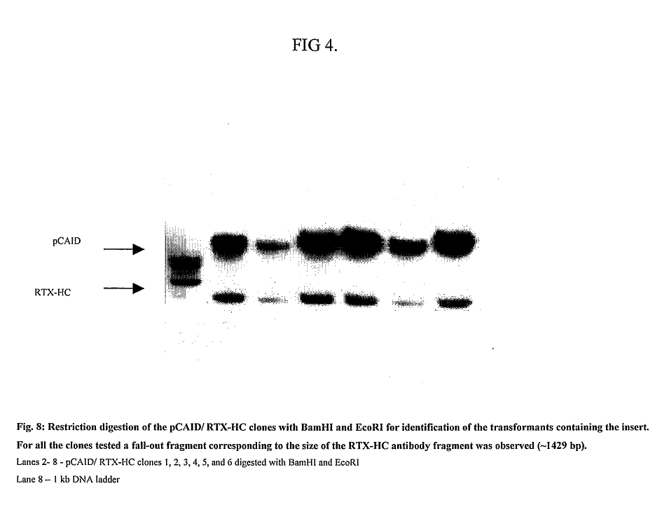

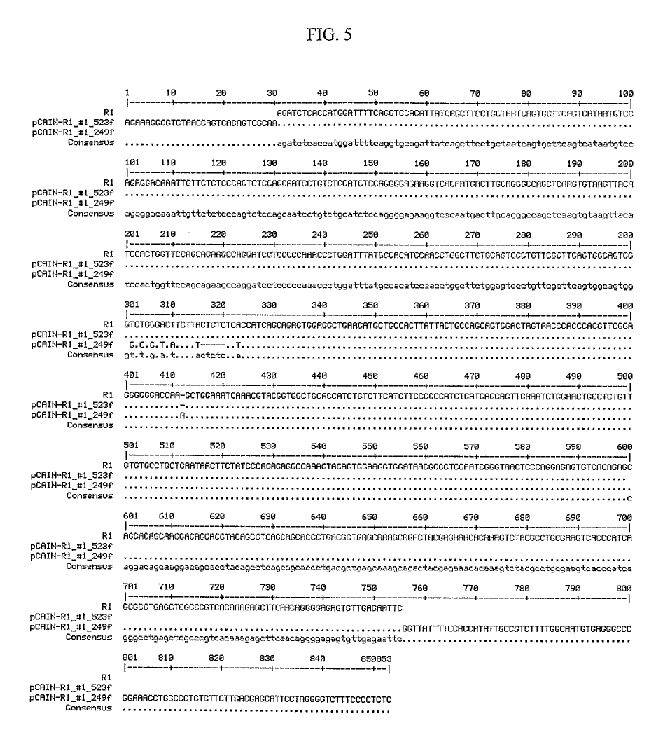

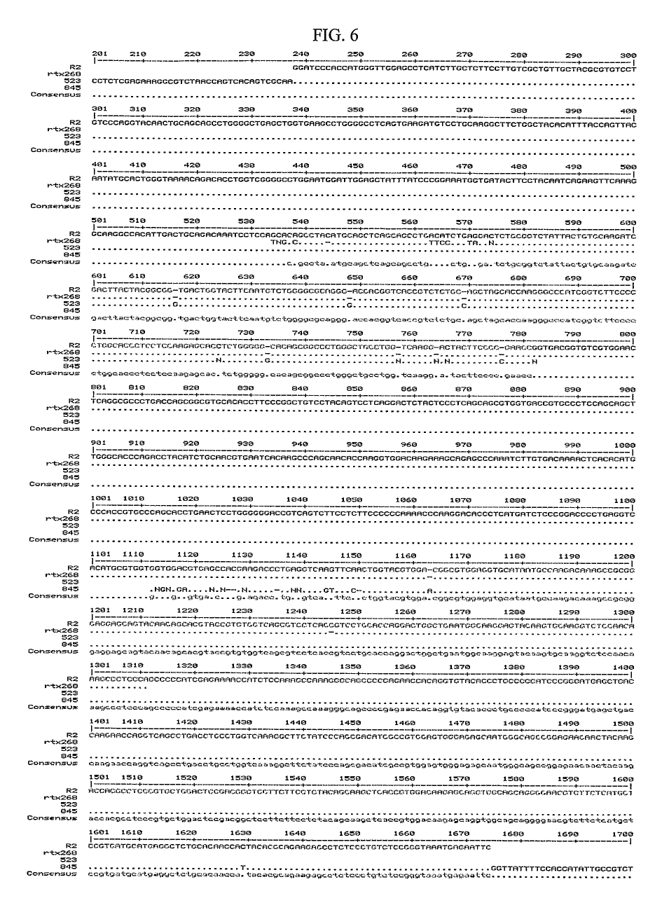

The full-length anti-CD20 antibody light and heavy chains were cloned sub-cloned into mammalian expression vectors pCAIN and pCAID respectively. The pBSKII/RTX-LC clone and the pCAIN vector were digested with Bg1II and EcoRI. The resultant construct referred to as pCAIN/RTX-LC. The insert (700 bp) from the former and the vector backbone from the latter were gel purified and ligated (FIG. 5). The ligation mix was transformed into heat shock competent DH10b cells and plated onto LB agar plates containing ampicillin as the selection antibiotic. A few transformants were picked and a DNA mini prep was carried out. The clones were checked by restriction enzyme digestion (FIG. 6).

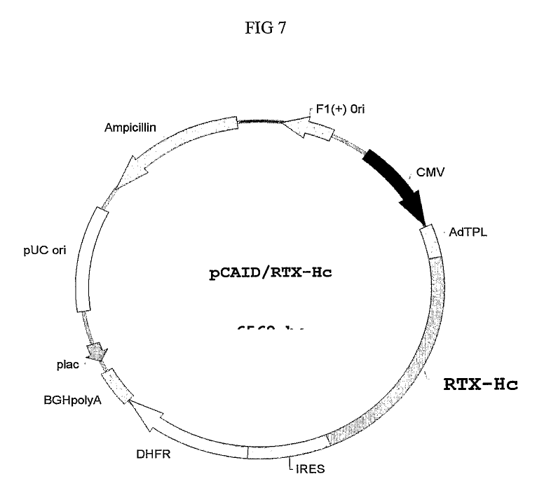

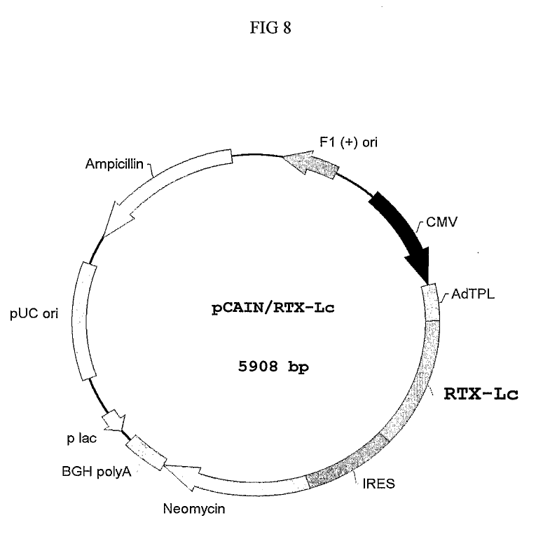

The pBSKII/RTX-HC clone and the pCAID vector were digested with BamHI and EcoRI and resultant construct referred as pCAID/RTX-HC. The insert (1429 bp) from the former and the vector backbone from the latter were gel purified and ligated (FIG. 7). The ligation mix was transformed into heat shock competent DH10b cells and plated onto LB agar plates containing ampicillin as the selection antibiotic. A few transformants were picked and a DNA mini prep was carried out. The clones were checked by restriction enzyme digestion (FIG. 8).

### DNA Sequencing and Analysis:

The final clones of RTX-HC and RTX-LC cloned into pCAID and pCAIN mammalian expression vectors respectively were sequenced and their sequence accuracy confirmed.

### Maintenance and Propagation of the Genes Encoding the Anti-CD20 Antibody Antibody Chains:

The cDNA construct encoding the chimeric light and heavy chain of the anti-CD20 antibody will be maintained and propagated in a standard bacterial cell line such as Top 10 (Invitrogen).

### Transient/Stable Recombinant Protein Expression in CHO-K1:

(a) Transient/stable expression of the construct will be done using the Chinese hamster ovary cells (CHO) a mammalian cell line that is FDA approved for industrial applications. Transient expression is useful to check the expression of a construct and to rapidly obtain small quantities of a recombinant protein.

(b) Subsequently, CHO cells that display a stable and high expression of the desired monoclonal antibody will be developed using standard procedures.

### Purification of the Anti-CD20 Antibody:

The mature chimeric antibody the anti-CD20 antibody is comprised of two antibody heavy chains (451×2 amino acids) and two antibody light chains (213×2 amino acids) and has an approximate molecular weight of 145 kDa. Both the chains have an N-terminal 20 amino acid leader sequence is cleaved off prior to the secretion of the hormone.

Subsequent to the establishment of reproducible bioactivity in accordance with the recommended functional/binding assays mentioned above, efforts will be made to optimize the purification procedures. The purification strategies will aim at process economics, speed to market, scalability, reproducibility, and maximum purity of the product with functional stability and structural integrity as the major objectives. To this effect, a combinatorial approach with both filtration (normal and tangential flow filtration) and chromatography would be explored. The process qualification requirements and acceptance criteria studies will be conducted on 3 batches.

Accordingly, the current invention envisages the following steps in the purification process:
a. Initial clarification using COHC/A1HC/0.45μ depth filters
b. Concentration using Pellicon XL Biomax 50 kDa cut-off filter based on tangential flow filtration
c. Chromo step—I: Affinity chromatography using Prosep VA Ultra for serum based (2% fetal calf serum [FCS])/and Prosep VA for serum free culture supernatants.
d. Chromo step—II: Strong cation exchanger such as SP Sepharose
e. Chromo step—III: Flow through based strong anion exchanger such as Cellufine Q (a cellulose based medium) for the removal of host cell proteins and nucleic acids.
f. Virus removal using size exclusion filtration and leached protein A using Cellufine sulfate
g. Sterile filtration
h. Endotoxin removal using either Remtox/Cellufine ET chromatography
h. Formulation

### Establishment of the Identity of the Target Protein Using Biochemical, Immunological and Physico-Chemical Methods:

The percent recovery of the total protein at each stage will be quantitated using bicinchoninic acid procedure (BCA)/Bradford dye binding method. The target protein concentration at each stage of purification will be probed using highly specific and reliable enzyme based immunoassays such as direct or indirect sandwich ELISA Qualitative and target specific western analysis will be followed at each stage. Reversed phase chromatography, isoelectric focusing and two-dimensional gel electrophoresis will be employed to evaluate the purified product. Secondary structural analysis would be examined using far UV circular dichroism. Molecular mass and oligomeric status will be investigated using size exclusion and MALDI-TOF. The investigations will also focus on the stability of the protein in relation to pH and temperature. As NESP is a hyperglycosylated protein, glycosylation patterns of the purified protein would be documented using gas chromatography (GC) analysis.

7. Assays for in vitro and in vivo Activity of the Anti-CD20 Antibody:

Bioassays for detecting in vitro CD20 binding of the anti-CD20 antibody and the effector function of the antibody will be done using:
a) Human C1q binding and CD-20 positive SB cells in a flow cytometry assay using fluoroscin labelled C1q
b) Complement dependent cell lysis of CD20 positive SB cells
c) Antibody dependent cellular cytotoxicity effector assay using CD20 positive cells and CD20 negative cells

Pre-clinical in vivo bioactivity of the anti-CD20 antibody will be tested on non-human primates (cynomolgus monkeys) for:
a) efficacy of B cell depletion from peripheral blood lymph nodes and bone marrow.
b) evaluation of any toxicity associated with the chimeric antibody

1. A process of preparing in vivo biologically active anti-CD 20 monoclonal antibody comprising the steps:
de novo synthesis of light and heavy chains of the anti-CD20 monoclonal antibody;
construction of full-length kappa light chain of the anti-CD 20 antibody;
construction of full length IgG1 heavy chain of the anti-CD 20 antibody;
construction of vectors comprising nucleic acid sequences encoding the light and the heavy polypeptide chains of the anti-CD 20 molecule; and
subcloning the anti-CD 20 antibody chains in a mammalian expression vector for production of the biologically active antibody molecule.

2. The method according to claim 1, wherein the nucleotide sequence encoding the light chain of the anti-CD20 antibody has been represented in SEQ ID NO: 1.

3. The method according to claim 1, wherein the nucleotide sequence encoding the heavy chain of the anti-CD 20 antibody has been represented in SEQ ID NO: 2.

4. The method according to claim 1, wherein the vector comprising the nucleic acid fragment encoding the heavy chain of the anti-CD20 is subjected to site-directed mutagenesis.

5. The method according to claim 1, wherein the full-length anti-CD20 heavy and light chain have been subcloned into a mammalian vector.

6. A method of preparation of an in vivo biologically active anti-CD20 monoclonal antibody comprising the steps of transforming a host cell with a vector construct of FIG. 7 or 8 and isolating the product from the host cell or the medium of its growth.

7. A pharmaceutical composition comprising a therapeutically effective amount of anti-CD20 antibody and a pharmaceutically acceptable diluent, adjuvant or carrier, wherein said antibody is purified from mammalian cells grown in culture.
